(19) United States
(12) Patent Application Publication
(10) Pub. No.: US 2003/0106099 A1
(43) Pub. Date: Jun. 5, 2003

(54) Unusual wheat starch mutants and amylopectin starch therefrom

(75) Inventors: Calvin F. Konzak, Pullman, WA (US); Rafiullah Sahibzada, Pullman, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Northwest Plant Breeding Co., Pullman, WA

(21) Appl. No.: 10/156,748

(22) Filed: May 28, 2002

Related U.S. Application Data
(63) Continuation-in-part of application No. 09/719,880, filed on Dec. 18, 2000.
(63) A 371 of international application No. PCT/US99/13801, filed on Jun. 18, 1999.
(60) Provisional application No. 60/293,753, filed on May 25, 2001.
(60) Provisional application No. 60/098,822, filed on Sep. 2, 1998.

(51) Int. Cl. A01H 5/00; C08B 31/00

(52) U.S. Cl. 800/284; 800/320.3; 536/102

(57) ABSTRACT

The present invention provides polyploid mutant wheat plants carrying at least one new mutation in a starch synthase gene, and progeny and seeds of such plants. The present invention further provides amylopectin starch from polyploid waxy wheat plants, the starch having new physical and chemical characteristics for food and industrial product uses.

### CONTINUITY

This application claims the benefit of U.S. Provisional Patent Application No. 60/293,753, filed May 25, 2001, and is a continuation-in-part of U.S. patent application Ser. No. ______, filed ______, which is a national stage patent application of Patent Cooperation Treaty Application No. PCT/US99/13801, filed Jun. 18, 1999, which claims the benefit of U.S. Provisional Patent Application No. 60/098,822, filed Jun. 19, 1998. All of these patent applications are incorporated by reference herein.

### BACKGROUND OF THE INVENTION

Plant starch is a co-polymer formed mainly of two components, amylose (or α-amylose) and amylopectin (or β-amylose); both components are glucose polymers. The glucose polymers differ in the degree of polymerization and in the degree of branching of the glucose chains. Amylose starch is a basically a non-branched polymer made up of α-1,4-glycosidic branched glucose molecules. Amylopectin starch is a mixture of more or less heavily branched glucose chains; the branching results from the occurrence of α-1,6-glycosidic interlinkages. Amylopectin starch can also contain phosphoric acid linkages and may have associated fatty acids.

The biochemical pathways that lead to starch synthesis are generally known. Enzymes involved in starch synthesis include starch synthases and branching enzymes. In the case of starch synthases, various isotypes have been described that catalyze a polymerization reaction by transferring a glucosyl residue of ADP-glucose to α-1,4-glucans. Branching enzymes catalyze the introduction of α-1,6 branchings into linear α-1,4-glucans. Starch synthases can be divided up in two groups: the granule-bound starch synthases (GBSS) and the soluble starch synthases (SSS). In cereals, GBSS control the process of debranching initially-formed amylopectin starch, leading to the formation of non-branched amylose starch. Plants in which GBSS activity has been strongly or completely reduced synthesize starch free, or substantially free, of amylose (i.e., “waxy” starch or amylopectin starch).

The wild-type Waxy genes (Wx) encode GBSS. Both natural and induced mutations of the genes encoding GBSS have been identified in many of the diploid species of cereals. Null or recessive mutants of the waxy gene locus result in the production of almost 100% amylopectin starch because the GBSS gene is non-functional or its function is altered (see, e.g., Briggs et al., Nature 207:891-92 (1965); Amano, Environ. Health Perspect. 37:35-41 (1981); Echt and Schwarz, Genetics 99:275-84 (1981)).

There is considerable interest within the food industry in mutant varieties of wheat that make altered starch. The physical properties and functionality of wheat flours are affected by the composition of the starch contained therein. For example, in foods, the “bite” or mouth feel, texture and palatability of Asian white noodles are adversely affected by a high amylose starch content. Conversely, mouth feel, texture and palatability of noodles are improved by an increase in the proportion of amylopectin starch. (See, e.g., Oda. et al., Cereal Chem. 57:253-54 (1980); Hoshino et al. U.S. Pat. No. 6,042,897.) Additionally, recent research has shown that amylopectin starch has a higher, longer water retention ability after cooking than starches containing more than 10% amylose. Because of this property, the baking industry has become interested in high amylopectin starch additions to, or proportions in, wheat flour to extend the shelf life of baked products. Amylopectin starch may increase the water-holding capacity, and thus extend the freshness retention of breads and other baked products. Many other uses of waxy (high amylopectin) wheat starches can be foreseen, including, but not limited to, uses in biodegradable plastics, thickeners, baby foods and puddings, and in glues which require lower amounts of emulsifiers and would likely have improved consumer reception. Further, the livestock industry is interested in obtaining waxy varieties of cereals for use in livestock feeds, because the greater branching structure of amylopectin starch makes the molecules more readily degradable by amylolytic enzymes, increasing the energy availability to monogastric animals. Thus, it is desirable to isolate variants of wheat which make starch lower in amylose and/or which create new forms of starch, allowing the development of new food and industrial applications.

Conventional methods for modifying the starch composition in plants are classical breeding methods and the induction of mutants. Naturally occurring waxy mutants are known in rice and maize. A mutant maize line also has been produced, which makes starch with an altered viscosity (see U.S. Pat. No. 5,331,108). A maize variety (waxy maize) was established by breeding; this waxy mutant produces starch consisting of almost 100% amylopectin starch (see Akasuka and Nelson, J. Biol. Chem. 241:2280-85 (1966)). Furthermore, mutants of potato and pea have been described which synthesize starches with a high amylose content (e.g., 70% in maize or up to 50% in pea). These mutants so far have not been characterized on the molecular level, and therefore do not allow for the induction of corresponding mutants in other starch-storing plants. Northwest Plant Breeding has recently identified high amylose accessions of T. aestivum and T. turgidum durum wheats, which have about 40% amylose, significantly higher than normal wheats, and similar to that present in a mutant of barley identified by Eslick et al.

Plant starch mutants also can be produced by recombinant DNA techniques. For example, the recombinant modification of potato plants aimed at altering the starch synthesized in these plants has been described (see, e.g., International Patent Cooperation Treaty Publications WO 92/11375 and WO 92/14827).

Naturally-occurring starch mutants have been more difficult to identify in polyploid species and are somewhat more difficult to produce by recombinant means, until gene sources present in the different genomes have been identified. The failure to identify naturally-occurring starch alleles in polyploid species has been primarily due to their recessive genetic nature and the requirement for independent mutations in the starch biosynthetic genes, one in each chromosome set/genome. For example, hexaploid wheat (Triticum aestivum L.) cultivars have three chromosome sets/genomes, derived from three different diploid species. The three chromosome sets of wheat are identified as three genomes, A, B and D. To develop a waxy hexaploid wheat, each of the three genomes would typically need to be homozygous for waxy alleles in each of the A, B and D chromosome sets/genomes. Similarly, durum wheat (Triticum turgidum durum) cultivars, which are tetraploid, also have not been observed to produce a naturally occurring waxy wheat, due to the redundancy of the GBSS genes in their A and B genomes.

Recently, it has become possible to identify and distinguish the enzyme protein bands produced by the GBSS genes in each of the three genomes in wheat by sodium dodecyl polyacrylamide electrophoresis (SDS PAGE). Plants having null or recessive natural mutant genes in one or more of their pairs of homoeologous genomes (A, B and D genomes) have been identified utilizing SDS PAGE (see, e.g., Chao et al., Theor. Appl. Genet. 78:495-504 (1989); Nakamura et al., Japanese Journal of Breeding 42:681-85 (1992)). Yamamori et al. (Euphytica 64:215-19 (1992)) used SDS PAGE to identify the three homoeologous GBSS isozymes encoded by the three homoeologous genomes of hexaploid wheat, via analyses for the presence or absence of the GBSS enzyme protein(s)in a large number of genetic lines and varieties. Chao et al. (supra) analyzed genetic stocks in which a pair of chromosomes, or part of a chromosome, was missing (i.e., nullisomic stocks, or ditelosomic stocks) using a cDNA probe of the waxy locus of barley for RFLP analyses of the GBSS loci in the chromosome sets/genomes of wheat. These analyses aided research by Nakamura et al. (Japanese Journal of Breeding 42:681-85 (1992)) using SDS PAGE to analyze over 1,800 wheat accessions from various parts of the world to identify genetic sources in which one or another of the homoeologous GBSS genes was inactive or deleted. Nakamura et al. (supra) were able to identify, among Japanese wheats, wheat accessions carrying a null mutant at each or both the A and B genome GBSS loci. They also found spontaneous null waxy mutations in the D genome of two old land race accessions from China, Bai Huo, a semi-spring wheat, and Bai Huo Mai, a semi-winter wheat. The identification of these spontaneous null waxy mutants permitted Nakamura and colleagues to develop fully waxy wheat (i.e., wheat, which produces essentially no GBSS), by recombination of the individual, homoeologous null loci to obtain T. aestivum and durum genotypes with near 100% waxy starch. Nakamura et al. also identified the varieties Kanto 79 and Kanto 107 as carrying null waxy mutations in both the A and B genomes, leaving the D genome with an active or dominant gene for the non-waxy trait.

Null waxy mutants are of limited use, however, in making new forms of starch, because they can only be combined in limited permutations. The properties of starch are mainly determined by its degree of branching, the amylose/amylopectin ratio, the average chain-length of the constituent polymers, and the occurrence of phosphate groups. Important functional properties of starch are, for example, solubility, tendency to retrogradation, capability of film formation, viscosity, water retention, color stability, pastification properties (i.e., binding and gluing properties), as well as resistance to alternating cold and warm storage periods, and amenability to the production of plastics for biorenewable products. The starch granule size also can be significant for the various uses. The disadvantage of the natural null alleles is that such alleles can only be recombined with other known waxy alleles in a limited number permutations, providing only limited variations in starch composition. Thus, there is a need for new starch mutants in wheat plants, including new waxy mutants, allowing for the production of other, even new, forms of starch, with often unusual physical and chemical properties.

### BRIEF SUMMARY OF THE INVENTION

The present invention relates to mutant polyploid wheat plants having mutant starch alleles that affect starch biosynthesis. Such mutant alleles can be at of the waxy loci or at other loci involved in starch biosynthesis.

In one aspect, a mutant polyploid waxy wheat plant, comprising at least two sets of chromosomes, is provided. Each set of chromosomes has a waxy locus, and a mutant waxy allele is present at each waxy locus. The waxy allele encodes a GBSS protein having altered enzymatic activity, as compared with wild-type GBSS protein. In one embodiment, the GBSS protein can have decreased enzymatic activity, as compared with wild-type GBSS protein. The polyploid wheat plant can be, for example, tetraploid or hexaploid. In another embodiment, each waxy locus comprises a waxy null allele or a mutant waxy allele encoding a partially active GBSS protein. Alternatively, each waxy locus can comprise a mutant waxy allele encoding a partially active GBSS protein. In certain embodiments, the GBSS protein can be modified, split (e.g., separated into different mutant genes, or truncated, relative to the wild type, full size GBSS protein. In additional embodiments, an additional mutation can be present in another starch synthesis locus.

In certain embodiments, the mutant polyploid waxy wheat plant can be derived from Bai Huo, Bai Huo Mai, Kanto 79, Kanto 107 or Ike. For example, the mutant polyploid waxy wheat plant can be K107 wx18, K107 wx21, K107 wx22, Ike wx 4, Ike wx 5A, or Ike wx 9A.

In another aspect, a mutant polyploid waxy wheat plant is provided which has one or more induced mutant waxy alleles. Some of the induced mutant waxy alleles encode a GBSS protein having an altered activity (e.g., a partially active GBSS protein). Progeny of the polyploid waxy plants are also provided. The progeny plants have waxy loci having waxy alleles encoding a GBSS protein(s) having altered activity (e.g., enzyme function). The waxy alleles can be the same or different. Starch granules from polyploid waxy wheat plants are also provided. A polyploid waxy wheat plant can be, for example, produced from a parent plant by mutagenesis with a mutagen to produce a mutant waxy gene from a wild-type gene, the mutant waxy gene encoding a GBSS protein having altered activity. The polyploid waxy wheat plant also can be reproduced to make progeny plants.

Waxy polyploid wheat seed is also provided, which comprises a mutant waxy gene encoding a GBSS protein having altered activity (e.g., enzyme function). The waxy polyploid wheat plant can also comprise at least one waxy allele encoding a partially active GBSS protein.

In another aspect, amylopectin starch from a mutant polyploid waxy wheat plant is provided. The amylopectin starch of the waxy seed can have a higher Brabender peak viscosity than amylopectin starch from other waxy mutants derived from Ike (PI 574488) or from other Kanto 107 waxy mutants, or from waxy wheats bred by recombining the naturally-occurring null GBSS alleles. In certain embodiments, the amylopectin starch can have a Brabender peak viscosity temperature of at least 68° C. In an exemplary embodiment, the amylopectin starch can have a peak viscosity of at least 1600 to about 1800 centipoise or about 1900 centipoise. In certain embodiments, the starch is from a mutant polyploid waxy wheat plant derived from Bai Huo, Bai Huo Mai, Kanto 79, Kanto 107 or Ike. For example, the mutant polyploid waxy wheat plant can be K107 wx18, K107 wx21, K107 wx22, Ike wx 4, Ike wx SA, or Ike wx 9A.

In another aspect, a mutant polyploid waxy wheat plant is provided which includes at least two sets of chromosomes, each set of chromosomes having waxy loci. The mutant polyploid waxy wheat plant may have independently induced mutant starch alleles present at each of the GBSS loci in the A and B or A, B, and D genomes, respectively, of the tetraploid or hexaploid wheat plant producing a starch having a higher Brabender peak viscosity than amylopectin starch from an isogenic strain having a wild-type allele at one or more loci of the different genomes. The amylopectin starch can have, for example, a Brabender peak viscosity temperature of at least 68° to 72 C., and/or a peak viscosity of at least 1600 to about 1800 centipoise, or about 1900 centipoise.

In certain embodiments, the mutant polyploid waxy wheat plant can be derived from Bai Huo, Bai Huo Mai, Kanto 79, Kanto 107 or Ike. For example, the mutant polyploid waxy wheat plant can be K107 wx18, K107 wx21, K107 wx22, Ike wx 4, Ike wx 5A, or Ike wx 9A.

### BRIEF DESCRIPTION OF THE DRAWINGS

FIG. 1 depicts the molecular weight distribution of starch polymers isolated from seeds of exemplary mutant polyploid waxy wheat plants designated Lab #III 1-10, which correspond to designations IKE Wx A, IKE Wx B, IKE Wx C, IKE Wx 1C, IKE Wx D, IKE Wx 9A, IKE Wx 3E, IKE Wx 2G, IKE Wx 4 and IKE Wx 5, respectively.

### DETAILED DESCRIPTION OF THE SPECIFIC EMBODIMENTS

The present invention relates to mutant polyploid wheat plants and to starch produced by such plants. The mutant polyploid wheat plants carry mutant alleles (mutant starch alleles) of genes involved in starch biosynthesis. The mutant starch alleles produce protein having altered function, as compared with the wild-type protein. The mutant starch alleles can be alleles of waxy loci or other loci involved in starch biosynthesis, such as amylopectin or amylose starch biosynthesis.

In one aspect, the mutant starch alleles are mutant waxy alleles. The waxy locus encodes granule bound starch synthase enzyme (GBSS). The full size GBSS protein has an apparent molecular weight of about 60 kilodaltons (kDa), as measured by SDS PAGE gel electrophoresis. Mutant alleles of the waxy locus (wary alleles) can affect the properties of starch produced by mutant polyploid wheat plants carrying those alleles. Mutant waxy starch alleles according to the present invention encode proteins having altered activity, as compared with the wild-type protein. As used herein, altered activity can include, for example, decreased or increased enzymatic activity (e.g., an altered K_{M}, K_{cat}, Kₐ, K_{D}, Vₘₐₓ, and the like), altered substrate specificity, and the like. The mutant waxy starch alleles retain at least some activity, however, as compared with wild-type GBSS protein, or other starch synthesis protein(s).

In certain embodiments, the mutant waxy alleles encode GBSS protein retaining partial enzymatic activity. As used herein, the term “partial activity” is in reference to the activity of the wild-type protein (e.g., GBSS or other protein(s) involved in starch biosynthesis). A partially active GBSS protein can exhibit decreased activity, as compared with the wild-type GBSS protein, but greater activity than null or non-functional protein. For example, a partially active GBSS protein can exhibit greater than 5, 10, 20, 30, 50, or 75, or up to about 90 percent of wild-type GBSS activity. A mutant GBSS enzyme can also produce amylopectin starch of a different polymer composition, with different numbers of branches, and/or with different segment lengths between branches, as compared with wild-type GBSS protein.

The mutant GBSS proteins encoded by the mutant waxy alleles can carry amino acid substitutions, deletions, insertions and/or can be truncated duplicated or otherwise modified. In certain embodiments, the partially active, GBSS protein can be truncated, being shorter, and/or duplicated compared to the full size wild-type GBSS protein. The mutant waxy alleles are typically recessive or semi-dominant, depending on the genetic background. In other embodiments, the mutant waxy alleles can be gain of function alleles (i.e., encoding a protein having a new or different function, as compared with wild-type protein), dominant alleles, duplications (i.e., the presence of additional copies of waxy genes, such as genes encoding mutant waxy alleles), and the like, that affect starch biosynthesis.

In certain embodiments, partially active, mutant GBSS proteins can exhibit altered electrophoretic mobility, as compared with wild-type GBSS protein. Altered electrophoretic mobility can indicate a change in primary amino acid sequence (e.g., an insertion, deletion, substitution, truncation or duplication), a change the secondary, tertiary and/or quaternary conformation of the protein, a change in protein structure, and the like. It will be appreciated by the skilled artisan, however, that in some cases there also may be no physical structure change notable by electrophoretic analyses (e.g., due to compensatory changes in the protein composition), although the function of the enzyme is altered. For example, a GBSS enzyme having an altered structure or mobility might less-effectively debranch amylopectin, or have a different function, resulting in the production of amylopectin starch molecules with different relative proportions of highly and less branched polymers, or of polymers with longer interbranch lengths. Changes in electrophoretic mobility can be determined, for example, by native gel electrophoresis, denaturing gel electrophoresis (e.g., SDS gel electrophoresis), 2-dimensional gel electrophoresis, and the like. (See, e.g., Nakamura et al., Mol Gen. Gen. 248:253-59 (1995); Fujita et al., Biochem. Genet. 34:403-13 (1996); Yamamori et al., Theor. Appl. Genet. 101:21-29 (2000); the disclosures of which are incorporated by reference herein.) General methods for the analysis of proteins are disclosed in, for example, Ausubel et al., Short Protocols in Molecular Biology, 4^{th} Ed. (John Wiley & Sons, Inc. (1999)) and Bollag et al., Protein Methods, 2^{nd} Ed. (John Wiley & Sons, Inc. (1996)).

In additional embodiments, mutant starch alleles according to the present invention can be mutant alleles of other genes involved in protein biosynthesis. Such genes can encode, for example, biosynthetic enzymes, regulatory proteins, modification proteins, or other gene products. Such mutant starch alleles can include, for example, mutations conferring altered activity or partially or altered enzymatic activity (as discussed supra). The mutant starch alleles also can be null alleles, gain of function alleles, recessive alleles, semi-dominant alleles, dominant alleles, dominant negative alleles that affect starch biosynthesis.

In another aspect, mutant wheat plants according to the present invention are provided. Such mutant wheat plants are polyploid, such as, for example, tetraploid or hexaploid. Tetraploid wheat strains have A and B genomes. Tetraploid mutant wheat plants within the scope of the present invention can be, for example, Triticum turgidum durum cultivars, or experimental lines. Similarly, hexaploid wheat strains have three genomes, A, B and D. Hexaploid mutant wheat plants within the scope of the present invention can be, for example, Triticum aestivum L. cultivars, or experimental lines.

Polyploid mutant wheat plants according to the present invention can carry mutant alleles induced separately in one or more genes involved in starch biosynthesis, then recombined by breeding. For example, in certain embodiments, mutant waxy wheat plants can have some mutant waxy alleles at a waxy locus (or at multiple waxy loci) in the A, B and/or D genomes (e.g., derived from Bai Huo, Bai Huo Mai, Kanto 79, Kanto 107, Ike, and the like). In other embodiments, mutant waxy wheat plants can have two, or three mutant starch alleles that affect starch biosynthesis. The mutant starch alleles can be similar or different alleles, and can be at different loci. In certain embodiments, the plants are heterozygous for mutant GBSS alleles, and can include different or the same pairs of homoeologous mutant alleles in their genomes. In some mutant waxy wheat plants, the remaining starch biosynthetic loci can null alleles (either producing no protein or non-functional proteins), or can encode partially active proteins, and the like.

Mutant wheat plants according to the present invention typically harbor induced mutations in a starch biosynthetic gene. An “induced” mutation is a non-natural mutation, typically produced by mutagenesis of polyploid wheat plants or plant parts thereof. A variety of plant parts, such as, for example, pollen, anthers, seeds, and the like, can be mutagenized. The wheat strain selected for mutagenesis can be, for example, a partially waxy strain, a partially waxy null strain (e.g., a single or double waxy null mutant, depending on ploidy level), and/or can carry other mutations at one waxy locus, or at one another locus involved in starch biosynthesis. In an exemplary embodiment, a waxy wheat plant can be formed by mutagenesis of a double null, hexaploid, partially waxy wheat strain, or a single null, tetraploid, partially waxy strain, wherein the wild-type target locus is in either the A, B, or D genome of a hexaploid wheat, or in either the A or B genome of tetraploid wheats.

The wheat plant or parts thereof can be mutagenized with any suitable mutagen. In certain embodiments, the seed is treated with mutagen(s). The amount of the plant parts to be mutagenized can be selected according to the desired number of “hits” in the genome, the screening efficiency, and the like. The mutagen can be, for example, one or more chemical and/or physical mutagens. Chemical agents include, but are not limited to, ethyl methanesulfonate (EMS) (see, e.g., Neuffer, Maize Genetic Newsletter 45:146 (1971)), diethyl sulfate (DES), nitroso-methyl or ethyl nitrosamines, EMS followed by azide (e.g., sodium or potassium azide) treatment (see, e.g., co-pending U.S. patent application Ser. No. 09/719,880, filed Dec. 18, 2000; the disclosure of which is incorporated by reference herein), nitrosoguanidine, N-methyl nitrosourea, or other alkylating agents, N-N-methyl glycine, and physical agents, such as electromagnetic radiation, X-rays, gamma rays, thermal or fast neutrons, and the like. Combinations of mutagens, either chemical and/or physical, can be employed. As will be appreciated by the skilled artisan, other mutagenic agents also can be used.

In an exemplary embodiment, seeds are presoaked (immersed in water) for 4-6 hours prior to mutagen treatment. After the presoaking period, the water is poured off, and distilled water is added to each container of seeds. The mutagen ethyl methane sulfonate (EMS) is applied to the seeds in the distilled water. The treatment applied can include one treatment at about 0.3 to about 0.4% EMS per liter of distilled water, or multiple treatments, involving more than one type of mutagen. The seeds are allowed to soak in the mutagen solution for about 2 hours with shaking to improve the contact of the seeds with the mutagen. After this treatment, the EMS solution is removed (e.g., poured off into a container containing sodium thiosulfate, and allowed to degrade before disposal), and 1 mM phosphate buffer (pH ˜3.5) is added to each container. Azide is added to the phosphate buffer solution to a final concentration of about 1.5 mM. The containers are shaken to distribute the azide solution, facilitating its absorption by the seeds. This azide treatment is continued with intermittent shaking for about 1 hour. After azide treatment, the azide solution is poured off into a container containing sodium thiosulfate and a soap and held for a week or more to degrade it, before disposal. The seeds are rinsed twice with water and allowed to dry at room temperature or cooler. The dried seeds are then planted as soon as possible, or the re-dried, treated seeds can be stored in a refrigerator (at 4 C.) for several days before planting, if necessary or desired. At maturity, the seeds from the crop can be harvested, cleaned of debris and readied for analysis.

Following mutagenesis, the seeds from plants grown from the treated plant parts/seeds and can be screened for mutant starch alleles, and the induced mutant starch produced can be characterized. For example, the seeds (e.g., the endosperm) can be screened for mutant starch alleles. In another example, seed can be mutagenized, and the mutagenized seeds germinated. The germinated plants (M1) can be allowed to self-fertilize, and M2 seed can be harvested. The M2 seed can be screened for mutant starch alleles.

The presence of mutant starch alleles can be determined by techniques known in the art (or to the skilled artisan), such as the analysis of starch, screening for changes in the individual wild-type GBSS proteins, at which the mutagenic treatment was aimed, and the like. For example, seeds can be screened for opaque-appearing grains; waxy seeds can have an opaque, lighter, coloration, as compared with non-waxy seeds).

Other assays for mutations affecting starch include screening seed or other plant parts for starch type and content. Waxy wheat starch typically stains red brown with iodine; this red brown stain identifies the starch as waxy starch. New starch mutants can be identified by changes in color after staining with iodine, as compared with a parental strain. For example, the posterior portion of the endosperm from seeds can be excised, and the embryo end of each seed can be tested with an iodine-potassium iodide solution (stock solution 3 grams Iodine (12) crystals: 15 grams potassium iodide (KI) crystals in 100 ml distilled water, diluted 1:10 with distilled water for the seed tests). Non-waxy seeds typically stain a dark blue color, while waxy (wx) seeds typically stain a reddish-brown color.

The screening process optionally can be automated, such as, for example, by using a double sanding belt apparatus that automatically scars the seed to expose the endosperm. The seeds can be passed between two sandpaper belts, which can be advanced by an electric motor device, causing the seed coats of the grains to be scratched enough to expose endosperm tissues. The scarred seeds can then be treated with an iodine (IKI) solution, and a short rinse to remove excess iodine and allow the starch to stain. The stained seeds are then allowed to dry (20 min-2 hours) with the grains spread on a surface for drying and subjected to visual observation for the seeds with reddish-color-stained endosperm. The grains having starch stained reddish-brown are the waxy grains. The method has proved highly efficient and far more certain than the seed opaqueness observation method, because waxy grains are difficult to identify from many soft textured, white-seeded, often starchy endosperm wheats.

In certain embodiments, protein assays can be used to confirm for the presence protein(s) having an altered electrophoretic mobility, such as, for example, a truncated GBSS protein, as discussed above. For example, because the full size GBSS protein is about 60 KDa, a modified protein may exhibit an apparent molecular wheat of more or less than 60 kDa, as determined by SDS PAGE gel electrophoresis, 2-dimensional gel electrophoresis, and the like. Similarly, a truncated protein can be detected as a protein having altered mobility, as compared to wild-type protein. Null alleles and other mutations can be detected by similar methods, as will be appreciated by the skilled artisan.

In other embodiments, immunoassays (e.g., Western blotting, immunoprecipitation, radioimmunoassay, ELISA (enzyme linked immunosorbent assay), “sandwich” immunoassay, precipitin reaction, gel diffusion precipitin reaction, immunodiffusion assay, agglutination assay, complement-fixation assay, immunoradiometric assay, fluorescent immunoassay, protein A immunoassay, and the like) can be used to detect mutant proteins. Such methods are generally disclosed, for example, by Ausubel et al., Short Protocols in Molecular Biology, 4^{th} Ed. (John Wiley & Sons, Inc. (1999)), Bollag et al., Protein Methods, 2^{nd} Ed. (John Wiley & Sons, Inc. (1996)), and Harlow and Lane, Using Antibodies, A Laboratory Manual (Cold Spring Harbor Laboratory, New York (1999)).

Mutant starch alleles can also be detected, for example, by DNA sequence analysis, polymerase chain reaction (PCR), and the like. (See, e.g., Yan and Bhave, Biochem. Genet. 38:391-411 (2000); Yan et al., Genome 43:264-72 (2000). See generally Ausubel et al., Current Protocols in Molecular Biology, 4th ed., John Wiley and Sons, New York (1999); Sambrook et al., Molecular Cloning, A Laboratory Manual, 3rd ed., Cold Spring Harbor Publish., Cold Spring Harbor, N.Y. (2001).) For example, nucleic acids at genetic loci involved in starch biosynthesis can be amplified by PCR from nucleic acids isolated from a seed of a plant grown from a mutagenized seed. Oligonucleotide primers representing known sequences of the starch locus can be used as primers in PCR. Alternatively, the oligonucleotide primers can represent at least a fragment of a conserved segment(s) of identity between starch loci of different plant species (e.g., conserved sequence between potato and wheat GBSS genes (see, e.g., U.S. Pat. No. 6,211,436)). Synthetic oligonucleotides can be utilized as primers to amplify particular oligonucleotides within a gene by PCR sequences. PCR can be carried out, for example, by use of a Perkin-Elmer Cetus thermal cycler and Taq polymerase (Gene Amp). Degenerate primers for use in the PCR reactions also can be synthesized. For example, the CODEHOP strategy of Rose et al. (Nucl. Acids Res. 26:1628-35 (1998), which is incorporated by reference herein) can be used to design degenerate PCR primers using multiply-aligned sequences as a reference. Methods for performing PCR and related methods are well known in the art. (See, e.g., U.S. Pat. Nos. 4,683,202; 4,683,195 and 4,800,159; Innis et al., PCR Protocols: A Guide to Methods and Applications, Academic Press, Inc., San Diego, Calif. (1989); Innis et al., PCR Applications: Protocols for Functional Genomics, Academic Press, Inc., San Diego, Calif. (1999); White (ed.), PCR Cloning Protocols: From Molecular Cloning to Genetic Engineering, Humana Press, (1996); EP 320 308; which are incorporated by reference herein in their entirety.) Starch gene expression can be assayed by, for example, screening for the presence of RNA from a locus involved in starch biosynthesis (e.g., by Northern blot assays, dot blots, in situ hybridization, quantitative reverse transcriptase-PCR, and the like).

Mutant alleles of starch biosynthetic genes can also be detected by enzyme assay. For example, partially active GBSS enzyme might less-effectively debranch amylopectin, or have an unusual mode of action, resulting in the production of amylopectin starch molecules with different relative proportions of highly and less branched polymers. Such an altered enzymatic activity can be detected by in vitro assay. (See, e.g., Nakamura et al., Plant Physiol. 118:451-59.)

Once detected, the waxy grains are selected, and plants can be grown from them to confirm their waxy nature. After confirmation, the waxy grains can be increased, in order to produce enough grain for detailed laboratory analyses of the starch properties.

Once isolated, polyploid wheat plants according to the present invention can be used for seed and/or starch production. Alternatively, the mutant starch alleles can be transferred by breeding or other techniques to form progeny polyploid wheat plants (e.g., progeny waxy wheat plants). In one aspect, the progeny plants are isogenic plants that have essentially identical genes as the parents, or as another desired strain. For example, isogenic waxy plants once produced can be bred through traditional breeding methods, or marker breeding methods, to move (i.e., transfer) specific waxy allele(s) into different wheat germ plasm, or to recombine different alleles to develop a new, reconstituted waxy wheat. The transmission of the waxy trait (or another desired trait) can be retained in the breeding process by, for example, monitoring the starch content of the progeny (e.g., iodine staining), by physical characteristics (e.g., by screening seeds for opaqueness), by monitoring the transmission of phenotypic markers genetically linked to the starch locus (e.g., chromosome 7 markers for A & D genome, or chromosome 4, for B genome waxy alleles, since the chromosome segment homoeologous to a part of the B genome chromosome 7 was long ago translocated to chromosome 4), by screening progeny directly for the presence of mutant starch alleles (e.g., by electrophoresis, PCR, RT-PCR, and the like), or other methods known to the skilled artisan.

In an exemplary embodiment, isogenic waxy wheat plants are prepared. For example, a polyploid (tetraploid) plant is selected (e.g., having at least one null waxy allele in one of its two sets of chromosomes, and a wild type allele in the other set). This seed is mutated, such as, for example, by EMS+azide mutagenesis (supra). Progeny seeds are screened for the desired waxy mutation in the wild type allele, which produces a partially active GBSS protein or otherwise produces starch with the desired properties. The presence of mutant GBSS protein can be confirmed by screening using electrophoresis, immunoassay, and the like, to detect the presence of a mutant form of the wild type GBSS protein. Plants, progeny plants, and/or seed can also be screened for phenotypic traits, such as, for example, starch type and content, starch characteristics, seed opaqueness, and the like. Once selected, the germ plasm can be increased to provide sufficient plant material for subsequent steps.

In another exemplary embodiment, a waxy wheat isogenic to the commercial variety Ike is induced to include a mutant starch allele of the wild type D-genome locus. Ike has been shown (Graybosch et al., Cereal Chemistry 75:162-65 (1998)) to carry recessive/null waxy alleles in its A and B genomes, and a ‘wild type’ Wx allele in its D genome. Seeds of this variety, obtained from the releasing State, Kansas, through the cooperation of Dr Joe Martin at Hays, Kans., can be mutagenized with EMS and sodium azide (supra). The number of treated seeds is typically large enough to produce the desired mutation events. The probable number of desired mutation events is a function of genetics (e.g., the Ike genotype is Awx, Bwx, DWx) ploidy, the amount and concentration of mutagen used, and the like. This number can be calculated by one of ordinary skill in the art. For example, Ike can be mutagenized so that the desired waxy mutant allele in the D genome occurs in about every 4,000-5000 seeds, or less.

The mutagenized seeds can be planted. The resulting wheat plants can be grown, fertilized, herbicide-treated and/or insecticide treated, as appropriate. When ready, the wheat is harvested, such as by hand or mechanical harvesting. The chaff of the spikes can be removed during the threshing process, and the seed readied for screening. The seeds are screened for the desired starch trait by viewing the seed on a light box, or by just sorting to identify opaque-appearing seeds. The seeds appearing opaque can be selected and stained with iodine staining after cutting with a razor blade and applying iodine solution to the endosperm portion of the seed. If the endosperm stains red-brown, the seed is waxy; if the endosperm stains blue, the seed is discarded, or could be planted to produce a second generation plants having M3 seed, from which to select more waxy mutants by the same methods. Some of the blue-staining seed may be heterozygous for waxy mutants not detectable in the seeds from the M1 generation plants. The red-brown stained seeds may carry a new waxy allele in the D genome, or other related starch mutant. After increasing the selected waxy seeds, the waxy starch mutant can then be analyzed by methods known in the art to characterize the mutant starch alleles.

In a related aspect, selected mutant starch alleles producing unusual types of starch can be recombined with other starch alleles, such as altered function alleles, null alleles, recessive alleles, loss of function alleles (e.g., null alleles or partially active alleles), modified gain of function alleles, alleles having decreased or altered function, semi-dominant alleles, dominant alleles, dominant negative alleles, duplications, and the like. In one embodiment, a mutant waxy allele is combined or recombined with other waxy alleles, including waxy null alleles, wild-type non-Waxy alleles, partially active waxy alleles, and the like. As an example, D genome mutant waxy alleles can be recombined by breeding or other methods (supra) with natural, or newly-induced waxy alleles in other genomes of polyploid wheat. A D-genome mutant waxy allele can be introduced into plants carrying other waxy genes in their homeologous A and B genomes. The A and B genomes can carry waxy null alleles, wild-type non-waxy alleles, or other waxy alleles according to the present invention. Similarly, new mutant waxy alleles according to the present invention can be induced in the A and/or B genomes and combined/recombined with a selected D genome mutant waxy alleles, to develop plants which produce new forms of starch.

In a related aspect, waxy starch (e.g., amylopectin starch) from plants carrying new combinations of starch mutants according to the present invention are provided. Waxy starch from such plants can have novel characteristics. The starch can differ from wild-type starch, or starch produced by single or double waxy null alleles, by the degree of branching, and/or by inter-branch lengths, amylopectin content, by amylose content, or apparent amylose content, by polymer size, and the like. In addition, the starch can have different physical/chemical characteristics, such as viscosity, gelling characteristics, size range of the starch granules and/or the shape of the starch granules.

New starch forms can be prepared by selecting combinations of mutant starch alleles according to the present invention and introducing those alleles into polyploid wheats. plants. Multiple null allele fully waxy wheat mutants, such as those derived by recombining waxy alleles among hexaploid wheats, like the American cultivar Ike or the Japanese wheat line Kanto 107, with the Chinese land races Bai Huo or Bai Huo Mai, typically produce near 100% amylopectin starches (having less than 5% apparent amylose). Mutant starch alleles according to the present invention can be introduced into such partial waxy null allele American/Japanese wheat varieties/lines; the resulting partial mutant polyploid wheat plants may have altered starch characteristics, as compared with starch from wild-type or parent plants, and/or the triple null waxy derivatives of the cross-combinations indicated above. For example, differences can be detected in the average size (molecular weight) of amylopectin starch, in the average size (molecular weight) of amylopectin starch, in the amylopectin/amylose ratio, in the branch ratio, in the long chain branch frequency, in viscosity, in gelling properties, and the like. Such differences can be detected using methodologies known to the skilled artisan. (See, e.g., Yamamori et al., Theor. Appl. Genet. 101:21-29 (2000); the disclosure of which is incorporated by reference herein.)

For example, the Brabender Micro Visco-Amylograph methodology can be used to compare starch viscosity and peak viscosity temperature. The analysis is generally directed to determining the pastification temperature, the maximum viscosity during heating, the increase in viscosity during cooling, as well as the viscosity after cooling. These parameters are important characteristics when it comes to the quality of a starch and its uses.

The Brabender test is carried out by using an appliance which is, for example, known as viscograph E. This equipment is produced and sold, among others, by C. W. Brabender Instruments, Inc. and Brabender OHG Duisburg (Germany). The test generally includes first heating starch in the presence of water in order to assess when hydration and the swelling of the starch granules takes place. This process, which is also named gelatinization or pastification, is based on dissolving hydrogen bonds and involves a measurable increase of the viscosity in the starch suspension. While further heating after gelatinization leads to the complete dissolving of the starch particles and to a decrease of viscosity, the immediate cooling after gelatinization typically leads to an increase in the viscosity. The result of the Brabender test is a graph which shows the viscosity depending on time, whereby at first the solution is heated to above the gelatinization temperature and then cooled. Typically, the starch paste viscosity is measured by holding at the peak viscosity temperature, usually 95° C. for 5 minutes, for non-waxy starches, then gradually decreasing the temperature of holding by 6.5° C./minute down to 50° C., and measuring the viscosity again.

Amylopectin starch produced by the wheat plants containing one or more mutant starch alleles according to the present invention (e.g., new waxy alleles) can have similar or higher peak viscosity temperatures (e.g., between 68° C. to 72° C. or higher), compared to waxy starches derived by recombining the naturally-occurring waxy genes from such germplasm stocks as Ike, or Kanto 107 x Bai Huo or Bai Huo Mai. Higher peak viscosity temperatures (e.g., greater than 68° C. peak viscosity temperature) can be due to the small differences in amounts of ‘apparent’ amylose, or to differences in the polymer sizes, branching and frequencies in the composition of the mutant starches. ‘Apparent’ amylose can include starch polymers with reduced branching and larger polymer sizes than those of ‘normal’ (i.e., natural) amylopectin starch polymers. These prime starches retain their relatively high paste viscosities over the test conditions, including the final viscosity level. The test results suggest that several new amylopectin starch mutant types described in this invention, can have greater gel stabilities also at refrigerator temperatures, as 4-6° C.

### EXAMPLES

The following examples are provided merely as illustrative of various aspects of the invention and shall not be construed to limit the invention in any way.

### Example 1

A waxy wheat strain derived from IKE is used to produce waxy alleles carrying partially active waxy alleles. The IKE variety was a double null waxy alleles (i.e., an Awx, Bwx (a,b) double null waxy wheat). Grain (or seeds) of IKE were mutagenized with EMS and sodium azide as follows:

The seeds (about 2.5 kg) were presoaked for 5-6 hours in distilled water prior to mutagenesis. After the presoaking period, the water was poured off, and a liter of distilled water was added to each container of seeds. The mutagen ethyl methane sulfonate (EMS) was applied to the seeds in the distilled water solution. EMS was applied at a concentration of 0.3 to about 0.4% v/v. Multiple treatments (repeated on the harvested seed prepared for planting the next season) can also be used. The seeds were allowed to soak in the mutagen solutions for about 2 hours with shaking of the treatment containers about every 10-15 minutes during treatment to improve the contact of the seeds with the mutagen. After this treatment, the EMS solution was poured off into a container used for disposal, and one liter of 1 mM phosphate buffer (pH 3.5) was added to each container. To each container with seeds and phosphate buffer, 1.5 milliliters of a 1.0 M stock solution of sodium azide was added, and the containers were then shaken to distribute the mutagen. This azide treatment was continued with intermittent shaking (every 10-15 minutes) for 1 hour, after which the solution was poured off into the disposal container, and the seeds in each container were given a distilled water rinse. After rinsing, the seeds were placed to redry in screen baskets in a fume hood, or on greenhouse benches. Redrying was continued for 14 hours, then the seeds were taken to another laboratory to continue drying at room temperature for another 24 hours. At this point, the treated seeds were placed in seeder magazines and planted in the field, much as any seeds would be sown for production.

Weed control during the growing season involved the use of standard herbicides. At maturity, the seed crop of each lot was harvested. The harvested seeds of each lot were cleaned of debris and readied for mutant identification and screening. Candidate recessive waxy (wx) mutants were selected from among others in the bulk M2 seed lots by their visual appearance (waxy seed typically has an opaque, lighter coloration). The posterior portion of the endosperms from selected candidate mutant seeds were cut, and the larger, embryo end of each was tested with an iodine-potassium iodide solution (stock solution 3 grams Iodine (12) crystals, 15 grams potassium iodide (KI) crystals in 100 ml distilled water: this solution, kept in a brown bottle away from light, diluted 1:10 with distilled water for the seed tests). Non-waxy seeds stained a dark blue color, while waxy (wx) seeds stained a reddish-brown color. Seeds with a yellow-berry condition (an environmentally influenced trait) are often confused with seeds, but the iodine stain test can be used to distinguish this condition from waxy mutants. Selection of candidate waxy seeds was later found to be more efficient, by use of an electric motorized device that drives a pair of narrow sanding belts, between which seeds are passed to have their seed coats scratched to reveal sections of endosperm tissue. After passing the seeds through the sanding belt device, the scratched seeds were placed in a net, and dipped for about 2-3 minutes in an MK1 staining solution, after which the bag of seeds was rinsed off, and the seeds placed in a thin, one seed layer on paper towels and allowed to dry for at least ½ day, but not longer than 2-3 days, after which the seeds were sorted to identify those having reddish-stained endosperm sectors. These seeds were then selected for confirmation of their waxy characteristic by cutting a posterior portion to expose a larger section of endosperm and the same stain test made to confirm the waxy trait. Then, those waxy seeds selected were sown for initial grain production in a greenhouse, then their waxy condition rechecked, after which the seeds were sown in rows or plots for increase one or two generations to produce sufficient grain for milling and starch separation for detailed chemical and physical property analyses.

Subsequent analysis of the waxy seeds identified some waxy seeds producing a modified or truncated GBSS protein. In other waxy seeds, a GBSS protein could not be detected in the expected gel location.

### Example 2

The seeds from 10 waxy mutants (derived from Ike), each carrying a waxy allele encoding new waxy alleles were analyzed for certain standard parameters and by the Brabender starch test. Table 1 identifies the 10 waxy Ike mutants and characteristics of whole seed produced by these Ike mutants.

TABLE 1
Analysis of Whole waxy Seeds from Ike Mutants

| Lab # | | Whole Seeds | | |
|---|---|---|---|---|
| Designation | Description | Moisture (%) | Ash (%) | NIRS |
| III-1 | IKE Wx A | 8.40 | 1.784 | 57.02 |
| III-2 | IKE Wx B | 8.48 | 1.846 | 56.89 |
| III-3 | IKE Wx C | 8.36 | 1.889 | 64.19 |
| III-4 | IKE Wx 1C | 8.42 | 1.817 | 62.83 |
| III-5 | IKE Wx D | 8.38 | 1.842 | 61.19 |
| III-6 | IKE Wx 9A | 8.55 | 1.892 | 68.21 |
| III-7 | IKE Wx 3E | 8.98 | 1.922 | 65.18 |
| III-8 | IKE Wx 2G | 8.61 | 1.820 | 55.43 |
| III-9 | IKE Wx 4 | 8.55 | 1.868 | 68.18 |
| III-10 | IKE Wx 5 | 8.60 | 1.948 | 62.40 |

Table 2 depicts an analysis of flour produced from the same waxy Ike strains.

TABLE 2

| | Flour | | | | |
|---|---|---|---|---|---|
| Designation | Moisture (%) | Protein (%) | Ash (%) | Mix.Time | W.Abs |
| IKE Wx A | 15.99 | 16.71 | 0.624 | 2:05 | 70 |
| IKE Wx B | 15.68 | 16.93 | 0.654 | 2:00 | 70 |
| IKE Wx C | 16.00 | 17.23 | 0.644 | 2:00 | 70 |
| IKE Wx 1C | 16.52 | 16.50 | 0.614 | 1:45 | 69 |
| IKE Wx D | 15.79 | 17.15 | 0.639 | 1:30 | 70 |
| IKE Wx 9A | 16.16 | 16.34 | 0.644 | 2:10 | 70 |
| IKE Wx 3E | 16.56 | 16.83 | 0.674 | 2:40 | 70 |
| IKE Wx 2G | 16.65 | 16.46 | 0.617 | 2:20 | 69 |
| IKE Wx 4 | 16.27 | 17.37 | 0.685 | 2:30 | 70 |
| IKE Wx 5 | 15.99 | 17.44 | 0.701 | 2:45 | 70 |

Table 3 depicts an analysis of the moisture content and amylose content of prime starch from the Ike mutants.

TABLE 3

| Designation | Moisture (%) | Amylose (%) |
|---|---|---|
| IKE Wx A | 8.88 | 0.463 |
| IKE Wx B | 12.41 | 0.380 |
| IKE Wx C | 10.54 | 0.545 |
| IKE Wx 1C | 9.54 | 0.380 |
| IKE Wx D | 10.13 | 0.463 |
| IKE Wx 9A | 8.72 | 1.124 |
| IKE Wx 3E | 9.47 | 1.496 |
| IKE Wx 2G | 10.26 | 0.793 |
| IKE Wx 4 | 8.89 | 0.876 |
| IKE Wx 5 | 9.37 | 2.281. |

Table 4 depicts a prime starch amylograph of starch from the same Ike mutant strains. The starch paste viscosity was measured by holding at 95° C. for 5 minutes, then gradually decreasing the temperature of holding by 6.5° C./minute down to 50° C., and measuring the viscosity again.

TABLE 4

| | | | | Viscosity at | |
|---|---|---|---|---|---|
| | | Viscosity 5 | Viscosity at End | End of | |
| | Peak | Minutes After | of Holding at | Holding at | |
| Designation | Viscosity. | Peak | Peak (95° C.) | 50° C. | Peak temp. |
| IKE Wx A | 1620 | 980 | 640 | 690 | 69 |
| IKE Wx B | 1660 | 1020 | 660 | 690 | 69 |
| IKE Wx C | 1710 | 980 | 650 | 690 | 69 |
| IKE Wx 1C | 1650 | 1010 | 640 | 690 | 68 |
| IKE Wx D | 1640 | 1000 | 660 | 700 | 69 |
| IKE Wx 9A | 1880 | 1220 | 790 | 840 | 70 |
| IKE Wx 3E | 1520 | 980 | 640 | 690 | 70 |
| IKE Wx 2G | 1560 | 970 | 650 | 700 | 70 |
| IKE Wx 4 | 1800 | 1000 | 740 | 680 | 70 |
| IKE Wx 5 | 1960 | 1330 | 860 | 900 | 71′ |

### Example 3

Kanto 107 waxy mutants (created and identified as generally set forth in Example 1) were analyzed with respect to various physical and biological parameters. Control A-wx B-wx D-Wx Kanto 107, as well as non-waxy varieties, were included in the analyses for comparisons, and a waxy derivative (Kanto 107/Bai Huo) line, carrying also the natural or spontaneous mutant D-genome null allele from Bai Huo, as well as the waxy genes from Kanto 107 was included as well. In the subsequent discussion of the data set forth in Tables 5 and 6, seed sample 0 is the original Kanto 107 variety which carries two waxy null gene loci; seed samples 1 and 4-25 are waxy mutants (Kanto 107 mutant 2 was lost during germination), all derived from mutagenized Kanto 107 seed; seed sample 3 is a partially-penetrant waxy mutant (i.e., a mutant which does not show the full waxy phenotype; seed sample 26 is a soft white, spring wheat, cv. Penawawa, which carries a single waxy null allele at the B genome wx locus; and seed sample 27 is a soft white, spring wheat. cv. Alpowa, which does not carry any waxy gene loci. Sample 28 is the partially waxy A-wx B-wx variety Ike.

Table 6 sets forth the characteristics of the prime starch extracted from flour milled from the foregoing wheat seed samples. The starch fractions were separated from the flour by a process developed by Czuchajowska and Pomeranz, Cereal Chem. 70:701-06, and as disclosed in U.S. Pat. No. 5,439,526 (the disclosures of which are incorporated by reference herein). Prime starch is the essentially pure starch fraction, containing only unbroken starch granules, and separated from the principal protein fractions in the flour; it has a very low ash content and is very low in protein. The prime starch still contains small amounts of phospholipids and a very minor fraction of proteins mostly associated with the starch granules (GBSS enzyme proteins). In Table 5, data are presented comparing the amylograph analyses of the prime starch fractions of the various wheats. The amylographic data were generated by standard techniques set forth in Shuey and Tripples (eds.), The Amylograph Handbook, Am. Assoc. Cereal Chem., St. Paul., Minn. (1980), which is incorporated herein by reference. The data include the temperature at which the peak (highest) viscosity occurs, the actual recorded viscosity of the solubilized starch at the peak temperature, the viscosity 5 minutes after the peak viscosity was reached, the viscosity at the end of a period of 30 minutes holding at the peak temperature, and at the end of a period of 30 minutes holding the gelled starch at 50 C. The starch paste viscosity was measured by holding at 95° C. for 5 minutes, then gradually decreasing the temperature of holding by 6.5° C./minute down to 50° C., and measuring the viscosity again. As noted earlier, the amylose content reported for the new mutants represents ‘apparent’ amylose, which may not be true amylose, as defined above.

TABLE 5
Analyses of Prime Starch

| | Yield | Moisture | Protein | Ash | Amylose |
|---|---|---|---|---|---|
| Name | (%) | (%) | (%) | (%) | (%) |
| Kanto 107* | 66.09 | 9.19 | 0.343 | 0.249 | 18.78 |
| K107 wx1 | 62.85 | 12.64 | 0.409 | 0.107 | 5.86 |
| K107 partial | 70.15 | 9.88 | 0.466 | 0.246 | 17.85 |
| wx | | | | | |
| K107 wx4 | 66.78 | 12.74 | 0.369 | 0.119 | 9.18 |
| K107 wx9 | 66.8 | 16.33 | 0.367 | 0.129 | 5.47 |
| K107 wx10 | 66.06 | 8.83 | 0.289 | 0.073 | 6.15 |
| K107 wx12 | 67.54 | 8.75 | 0.270 | 0.076 | 5.29 |
| K107 wx13 | 67.05 | 10.43 | 0.342 | 0.070 | 5.87 |
| K107 wx14 | 64.38 | 8.93 | 0.239 | 0.072 | 5.67 |
| K107 wx15 | 61.77 | 11.39 | 0.275 | 0.040 | 5.46 |
| K107 wx16 | 64.93 | 11.69 | 0.305 | 0.040 | 5.57 |
| K107 wx18 | 55.79 | 9.24 | 0.298 | 0.084 | 5.85 |
| K107 wx20 | | | | | |
| K107 wx21 | 56.22 | 9.22 | 0.346 | 0.194 | 5.85 |
| K107 wx22 | 64.03 | 9.43 | 0.270 | 0.184 | 8.32 |
| K107 wx25 | 65.7 | 9.66 | 0.363 | 0.120 | 6.04 |
| Penawawa (1 | 76.24 | 10.39 | 0.380 | 0.191 | 19.93 |
| wx) | | | | | |
| Alpowa (Wx) | 77.62 | 9.56 | 0.367 | 0.192 | 21.93 |
| Ike* | 64.0 | 8.64 | 0.370 | | |

*As noted earlier, Kanto 107 and Ike are controls, each having A-wx, B-wx, D-Wx. Alpowa carries no recessive waxy alleles

Table 6 depicts the results from the Brabender tests on the prime starch the same K107 and other strains listed in Table 5. As can be seen, certain strains, such as 18, 21 and 22 exhibit peak viscosity temperatures of between 68° C. and 72° C. Higher peak viscosity temperatures (e.g., greater than 68° C. peak viscosity temperature) can be due to the small differences in amounts of ‘apparent’ amylose, or to differences in the polymer sizes, branching and frequencies in the composition of the mutant starches. ‘Apparent’ amylose, can include starch polymers with reduced branching and larger polymer sizes than those of ‘normal’ (i.e., natural) amylopectin starch polymers, more like, but different from ‘normal’, unbranched, amylose molecules. In addition, the starch from these strains exhibits higher peak viscosities and have comparatively higher paste viscosities over the test conditions, including the final viscosity level.

The test results indicate that these strains produce new amylopectin starch types. The data demonstrate differences among the mutants and the standards. Kanto 107 mutant 3 appears to be a partially waxy line, but it was first identified as waxy, like all the rest. The seed harvested from the M2 and M3 plants of Kanto 107 mutant 3 proved not to stain red like typical waxy mutants, thus was thought to have been selected in error. The seed of this mutant was increased anyway in order to have a single line selection from Kanto 107. However, as the amylograph data show, mutant 3 has properties similar or intermediate between those of Kanto 107 (A-wx, B-wx, D-Wx) and the truly waxy mutants 1 and 4-25. Mutant 3 contains a high amount of amylose, since its peak gelatinization temperature is much like that of Penawawa, though its peak viscosity is more nearly like that of Kanto 107, yet it has the lowest ending viscosity of the entire series of mutants and controls! Its amylopectin content is presumably intermediate between that of Kanto 107 and a fully waxy mutant, having nearly 99% amylopectin starch. Of special interest are the peak viscosity data for mutants 18 and 21. The gels of these mutants have an exceptionally high viscosity at their peak temperature of gelling. In addition, Kanto wx22 has an initial peak viscosity of over 1600, and after holding at that temperature and after storage at 50° C., it has the highest viscosity of all of the mutants (See Table 6).

TABLE 6
Prime Starch Amylograph for K 107 Strains

| | | | | Viscosity at | Viscosity at |
|---|---|---|---|---|---|
| | | | Viscosity 5 | End of | End of |
| | Temp. of | | Minutes | Holding at | Holding at |
| Name | Peak (° C.) | Peak Viscosity | After Peak | Peak | 50° C. |
| Kanto 107* | 93.5 | 1040 | 860 | 620 | 1040 |
| K107 wx1 | 70.0 | 1540 | 1000 | 680 | 650 |
| K107 partial | 95.0 | 930 | 740 | 520 | 490 |
| wx | | | | | |
| K107 wx4 | 71.0 | 1540 | 1160 | 860 | 830 |
| K107 wx9 | 68.5 | 1580 | 1040 | 670 | 720 |
| K107 wx10 | 69.0 | 1500 | 1000 | 640 | 690 |
| K107 wx12 | 68.0 | 1490 | 1000 | 660 | 680 |
| K107 wx13 | 68.0 | 1480 | 960 | 610 | 620 |
| K107 wx14 | 68.0 | 1500 | 1000 | 640 | 670 |
| K107 wx15 | 67.5 | 1500 | 1000 | 660 | 670 |
| K107 wx16 | 68.0 | 1430 | 900 | 610 | 650 |
| K107 wx18 | 68.0 | 1800 | 1610 | 720 | 720 |
| K107 wx20 | | | | | |
| K107 wx21 | 67.0 | 1880 | 1200 | 740 | 730 |
| K107 wx22 | 72.0 | 1620 | 1450 | 1300 | 1210 |
| K107 wx25 | 68.0 | 1500 | 1010 | 650 | 670 |
| Penawawa | 95.0 | 680 | 980 | 620 | 610 |
| (1 wx) | | | | | |
| Alpowa | 96.0 | 360 | 390 | 450 | 1040 |
| (Wx) | | | | | |
| Ike 28* | 95.0 | 900 | 720 | 600 | 760 |

*As noted earlier, Kanto 107 and Ike are controls, each having A-wx, B-wx, D-Wx. Alpowa carries no recessive waxy alleles.

### Example 5

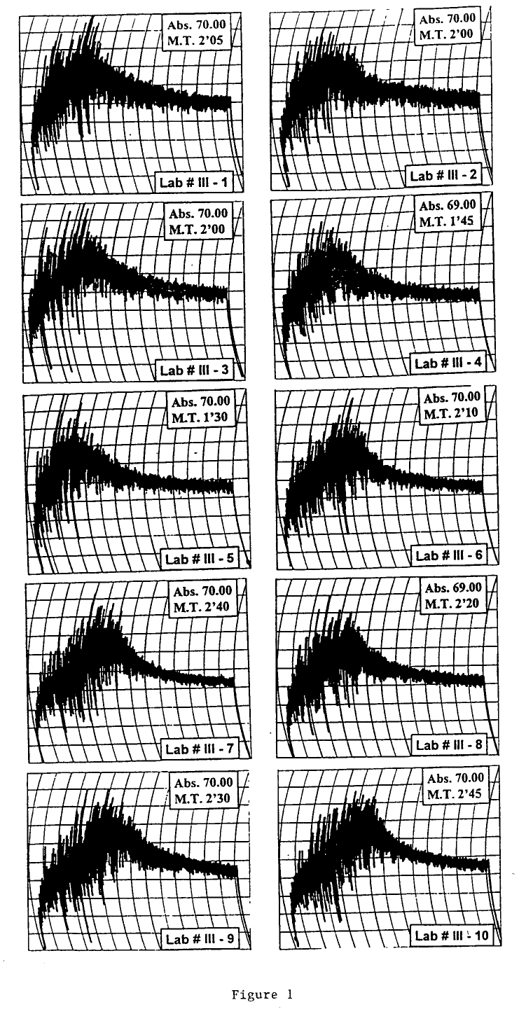

Starch isolated from partially waxy mutant plants, Bai Huo and J2297, carrying only a waxy D genome natural mutant waxy allele indicates that the starch has different ranges of polymer size populations than that of starch isolated from other plants. (See FIG. 1 and Table 7.) The differences in polymer size distributions of the starch is not as large as might be found in diploid species, largely, because all of the mutants from each of the two cultivars also carry the same two null waxy alleles in their A and B genomes.

Briefly, slurries (˜0.75% w/v) were prepared using aqueous 90% (v/v) methyl sulfoxide. Each slurry (10 ml) was heated at 100° C. for 45 minutes and stored 60° C. for 48 hours. Prior to storage for 48 hours, 0.1 ml of 3,5-dinitro salicylic (0.1N) was added to precipitate proteins. After storage, samples were subsequently centrifuged at 3,000×g for 10 min. Each supernatant was decanted and then filtered using a 1.2 μm filter. After filtration 1 ml aliquots were transferred to vials, and mixed with 90-100 mg of BioGel resin beads to remove any ions in solution. The vials were swirled in a shaking water bath for 30 min at room temperature. Samples (25 μl) were subsequently injected into the HPSEC system.

TABLE 7
Molecular weight distribution characteristics^{a} (M_{z},
M_{w}, and Mₙ), ratio and branching parameters of amylopectin and amylose in wheat samples.

| | Amylopectin (AP) × | | | | Amylose (AM) × | | | | AP/AM | | |
|---|---|---|---|---|---|---|---|---|---|---|---|
| | 10⁷ | | | | 10⁶ | | | | Branch LCBF^{b} | | |
| Sample | M_{z}^{c} | M_{w}^{d} | Mₙ^{e} | M_{w}/Mₙ | M_{z}^{c} | M_{w}^{d} | Mₙ^{e} | M_{w}/Mₙ | ratio | | |
| IkeControl* | 2492 | 2.072 | 1.738 | 1.2 | 5.667 | 3.87 | 2.900 | 1.3 | 59.0 | 6.4 | 1.9 |
| IkeWx1 | 3.067 | 2.503 | 1.738 | 1.4 | — | — | — | — | — | 13.2 | 0.1 |
| IkeWx2 | 2.928 | 1.453 | 1.800 | 1.4 | — | — | — | — | — | 6.0 | 0.2 |
| IkeWx3 | 3.737 | 3.121 | 2.309 | 1.4 | — | — | — | — | — | 9.3 | 0.1 |
| IkeWx4 | 2.831 | 2.275 | 1.569 | 1.5 | — | — | — | — | — | 8.8 | 0.2 |
| Bai Huo | 2.299 | 2.012 | 1.649 | 1.2 | 4.396 | 3.026 | 2.318 | 1.3 | 62.0 | 8.0 | 0.1 |
| J2297 | 3.569 | 3.136 | 2.459 | 1.3 | 9.745 | 5.673 | 4.141 | 1.4 | 62.0 | 10.0 | 0.1 |
| Kanto 107* | 2.985 | 1.734 | 1.174 | 1.5 | 6.789 | 4.9740 | 4.123 | 1.2 | 72.0 | 13.3 | 0.1 |
| Kantowx1 | 1.438 | 1.184 | 0.831 | 1.3 | 3.749 | 3.153 | 2.787 | 1.2 | 79.0 | 3.1 | 4.6 |
| Kantowx3 | 3.850 | 3.625 | 3.357 | 1.1 | 2.300 | 1.887 | 1.651 | 1.2 | 80.0 | 3.3 | 5.9 |
| Kantowx4 | 3.059 | 2.681 | 2.230 | 1.2 | — | — | — | — | — | — | — |
| Kantowx5 | 5.546 | 4.141 | 2.699 | 1.5 | — | — | — | — | — | 1.5 | 0.1 |
| Kantowx6 | 5.694 | 5.088 | 4.384 | 1.2 | — | — | — | — | — | 15.6 | 3.3 |
| Kantowx7 | 2.167 | 1.985 | 1.780 | 1.1 | — | — | — | — | — | 2.8 | 1.6 |
| Kantowx8 | 1.666 | 1.429 | 1.126 | 1.3 | — | — | — | — | — | — | — |
| Kantowx9 | 3.022 | 2.627 | 2.120 | 1.2 | — | — | — | — | — | 3.3 | 0.5 |
| Kantowx10 | 5.047 | 4.334 | 3.360 | 1.3 | — | — | — | — | — | 6.6 | 0.8 |
| Kantowx11 | 2.849 | 2.426 | 1.977 | 1.2 | — | — | — | — | — | — | — |
| Kantowx12 | 2.004 | 1.789 | 1.500 | 1.2 | — | — | — | — | — | 12.0 | 0.2 |
| Kantowx13 | 2.639 | 2.302 | 1.886 | 1.2 | — | — | — | — | — | 1.3 | 2.7 |
| Kantowx14 | 3.940 | 3.340 | 2.632 | 1.3 | — | — | — | — | — | — | 0.02 |
| Kantowx15 | 2.740 | 2.294 | 1.690 | 1.5 | — | — | — | — | — | — | — |
| Kantowx16 | 4.070 | 3.495 | 2.673 | 1.3 | — | — | — | — | — | 10.1 | 0.02 |
| Kantowx17 | 3.827 | 2.785 | 1.900 | 1.5 | — | — | — | — | — | 12.1 | 0.01 |
| Kantowx18 | 3.793 | 3.235 | 2.566 | 1.3 | — | — | — | — | — | 7.1 | 0.1 |
| Kantowx19 | 4.715 | 4.049 | 3.348 | 1.2 | — | — | — | — | — | 7.2 | 0.2 |
| Kantowx20 | 3.766 | 3.183 | 2.404 | 1.3 | — | — | — | — | — | 10.1 | 45.7 |
| Kantowx21 | 0.919 | 0.768 | 0.603 | 1.3 | — | — | — | — | — | — | — |
| Kantowx22 | 2.188 | 1.398 | 1.115 | 1.3 | — | — | — | — | — | — | — |
| Kantowx23 | 2.768 | 2.057 | 1.443 | 1.3 | — | — | — | — | — | — | — |
| Kantowx24 | 4.043 | 3.322 | 2.445 | 14 | — | — | — | — | — | 8.6 | 0.1 |
| Kantowx25 | 3.623 | 2.464 | 1.890 | 1.3 | — | — | — | — | — | 3.4 | 0.7 |
| Kantowx26 | 2.271 | 1.968 | 1.596 | 1.2 | — | — | — | — | — | — | — |
| Kantowx27 | 3.273 | 2.740 | 2.051 | 1.3 | — | — | — | — | — | 9.6 | 0.1 |

^{a}Values are means of two analyses.
^{b}Percent of branched polymers/material in starch.
^{c}Long chain branch frequency; number of branch points per polymer of 1,000 glucose units.
^{d}z-average molecular weight (Flex life, stiffness).
^{e}Weight-average molecular weight (Tensile strength, hardness).
^{f}Number-average molecular weight (Brittleness, flow properties).
^{g}Polydispersity.
*Controls

### Example 6

To determine whether the Ike and Kanto mutant wheat plants produced altered GBSS proteins, SDS PAGE was performed on granule-bound proteins isolated from starch granules. Ike mutants 5A and 9A produced a detectable GBSS protein. GBSS protein from IKE mutant 9A was smaller than wild-type GBSS protein. No protein was detected from Ike mutant 4, within the limits of this SDS page system, although this mutant plant likely expresses an altered GBSS protein, because it produces starch having unusual characteristics. Kanto mutants 22, 25, 26, 29, 38, 42 and 44 produced a detactable GBSS protein. GBSS protein from Kanto mutant 22 was smaller than wild-type GBSS protein. Kanto mutants 27, 30, 33-37, 39-41 and 43 did not produce a detectable GBSS protein, within the limits of this SDS page system. No protein was detected from Kanto mutants 18 and 21, within the limits of this SDS page system, although these mutant plants likely express an altered GBSS protein, because they produce starch having unusual characteristics.

These examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

What is claimed is:

1. A polyploid waxy wheat plant, comprising:
at least two sets of chromosomes, each set of chromosomes having waxy loci; and
a mutant waxy allele present at the waxy loci, the mutant waxy allele encoding a GBSS protein having altered function as compared with wild-type GBSS protein;
the wheat plant producing altered starch as compared with an isogenic wheat plant without the mutant waxy allele.

2. The wheat plant of claim 1, which is tetraploid.

3. The wheat plant of claim 1, which is hexaploid.

4. The wheat plant of claim 1, wherein at least one waxy locus comprises an induced mutant waxy null allele or an induced mutant waxy allele encoding a partially active GBSS protein, and the other waxy loci are induced waxy mutant alleles or naturally-occurring waxy alleles.

5. The wheat plant of claim 1, which is derived from Bai Huo, Bai Huo Mai, Kanto 79, Kanto 107 or Ike.

6. The wheat plant of claim 2, wherein the at least one induced GBSS protein is truncated, or modified, as compared with the wild-type GBSS protein of the same genome.

7. The wheat plant of claim 1, which is K107 wx18, K107 wx21, K107 wx22, Ike wx 4, Ike wx SA, or Ike wx 9A.

8. Seed from the polyploid waxy wheat plant of claim 1, the seed comprising at least one induced waxy allele encoding a partially active GBSS protein.

9. Amylopectin starch from the polyploid waxy wheat plant of claim 1.

10. Progeny from the polyploid waxy wheat plant of claim 1.

11. Amylopectin starch from the polyploid waxy wheat plant of claim 10.

12. A polyploid waxy wheat plant; comprising:
at least one induced mutation in at least one waxy gene (an induced waxy allele) encoding a partially active GBSS protein.

13. Progeny of the plant of claim 12, comprising waxy loci having the induced waxy allele encoding partially active GBSS protein.

14. Seed from the progeny of claim 13, the seed comprising at least one induced waxy allele encoding partially active GBSS proteins.

15. A waxy wheat starch granule from the plant of claim 1.

16. Amylopectin starch from the polyploid waxy wheat plant of claim 12.

17. Amylopectin starch from the polyploid waxy wheat plant of claim 13.

18. A polyploid waxy wheat plant which has been produced from a parent plant by mutagenesis with a mutagen to produce a mutation in a non-waxy (Wx) gene which encodes a partially active GBSS protein.

19. Waxy progeny of the plant of claim 18.

20. Seed from the polyploid waxy wheat plant of claim 18, the seed comprising at least one induced waxy allele encoding a partially active GBSS protein.

21. A waxy polyploid wheat seed, comprising at least one induced mutant waxy gene encoding a partially active GBSS protein.

22. Amylopectin starch from the polyploid waxy wheat plant of claim 18.

23. Prime starch according to claim 22, having a higher peak viscosity than prime starch from an Ike or a Kanto 107 double null waxy wheat plant.

24. Prime starch according to claim 23 from a fully waxy wheat plant, carrying only non-natural, induced mutant waxy alleles in its three genomes.

25. The amylopectin starch of claim 23, which has a peak viscosity temperature of at least 68° C.

26. The amylopectin starch of claim 23, having a peak viscosity of at least 1600 centipoise.

27. A mutant polyploid waxy wheat plant, comprising:
at least two sets of chromosomes, each set of chromosomes having waxy loci; and
an induced mutant starch allele present at each waxy locus;
the mutant polyploid wheat plant producing a starch having higher peak viscosity than prime starch from an isogenic strain having a wild-type allele at one or more waxy loci, or than amylopectin of prime starch from a fully waxy wheat carrying natural mutant alleles in its two or three genomes.

28. Amylopectin starch derived from the mutant polyploid waxy wheat plant of claim 27, which has a peak viscosity temperature of at least 68° C.

29. Amylopectin starch derived from the mutant polyploid waxy wheat plant of claim 27, having a peak viscosity of at least 1600 centipoise.

30. Amylopectin starch derived from the mutant polyploid waxy wheat plant of claim of claim 27, wherein the mutant polyploid waxy wheat plant is K107 wx18, K107 wx21, K107 wx22, Ike wx 4, Ike wx 5A, Ike wx 9A, or a mutant or progeny thereof.
